(19) United States
(12) Patent Application Publication
(10) Pub. No.: US 2008/0058748 A1
(43) Pub. Date: Mar. 6, 2008

(54) Disposable absorbent article having a graphic adapted to facilitate discretionary use of said article

(76) Inventors: Kathy P. Seifert, Appleton, WI (US); Rebecca S. Walter, Hortonville, WI (US); Garry R. Woltman, Appleton, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/511,725

(22) Filed: Aug. 29, 2006

(51) Int. Cl. A61F 13/15

(52) U.S. Cl. 604/385.01

(57) ABSTRACT

Disclosed is a disposable absorbent article having a graphic disposed thereon adapted to promote discreet use of said article when used in proximity with a second article of manufacture. Also disclosed are product lines comprising a plurality of designators, such as SKUs, with each designator corresponding to a disposable absorbent article having a different graphic disposed thereon so that a consumer or user can select an article having a graphic that is the same or similar to a second graphic disposed on a second article of manufacture that will be proximate to said disposable absorbent article during use of said article.

### BACKGROUND

People rely on disposable absorbent articles, such as disposable diapers for infants, disposable training pants for toddlers, and incontinence products for adults, as part of their everyday lives.

Disposable absorbent articles are designed to absorb and contain body wastes. Caregivers use disposable absorbent articles for several reasons, including the convenience of the caregiver, and the health and comfort of the wearer of the disposable absorbent article. Accordingly, manufacturers of disposable absorbent articles spend considerable amounts of time and money on researching and developing: new disposable absorbent articles; new features for disposable absorbent articles; improved performance of existing disposable absorbent articles; and other related research-and-development activities. Manufacturers also spend significant sums of time and money communicating the existence and/or benefits of such new and/or improved disposable absorbent articles to consumers through advertising, packaging, and other marketing activities.

Some users or caregivers may be embarrassed by the need to employ various disposable absorbent articles in various contexts in which the user interacts with others. For example, an adult having incontinence problems may need to use disposable absorbent undergarments, disposable absorbent pads for use with a woven undergarment, disposable absorbent bed pads, disposable absorbent furniture pads, and the like. If the adult is active, then he or she may employ such articles in contexts outside the home, perhaps in sports, camping, etc. An adult may wish to be, where possible, discreet about using such disposable absorbent articles.

In another context, a younger person may have trouble wetting the bed at night (e.g. because of a bladder problem). Accordingly, such persons and/or their caregivers may need to employ disposable absorbent articles in a variety of contexts, including those identified in the preceding paragraph. Other children may lack bladder control generally, and, like the adults mentioned above, may need to employ disposable absorbent articles in many different settings, e.g., sports, camping, and the like. Again, these younger persons may be embarrassed about using such disposable absorbent articles. They and their caregivers (e.g., parents or guardians) may seek out disposable absorbent articles that facilitate discretionary use of such articles.

Often disposable absorbent articles are available that do not blend in with other articles proximate to said disposable absorbent products during use. Furthermore, it appears that manufacturers and retailers of said disposable absorbent articles do not provide a plurality of stock-keeping units (SKUs) (or other designator of a product or products in a product line), each corresponding to a disposable absorbent article having a different graphic disposed thereon, so that users or caregivers may select an article bearing a graphic that will blend with, or be the same as, or similar to, or be coordinated with graphic(s) on other articles proximate to said disposable absorbent article during use of the disposable absorbent article. For the same reason, manufacturers and retailers of said disposable absorbent products have not developed messages embodied in tangible media (e.g., the packaging containing said disposable absorbent articles) that make users and/or caregivers aware of promoting discretionary use of said products through selection of a disposable absorbent article having a graphic disposed thereon that blends in with, is the same or similar to, or coordinates with the graphics or appearance of an article that will be proximate to said disposable absorbent article during use of the disposable absorbent article. Also, manufacturers and retailers appear not to have identified and made available kits that include one or more disposable absorbent articles and at least one second article of manufacture adapted to be used with said disposable absorbent article, wherein both the disposable absorbent article and the second article of manufacture have the same or similar graphics, or a common theme, or coordinated graphics, such that use of the disposable absorbent article together with the second article of manufacture promotes discretionary use of said disposable absorbent article.

What is needed is: a disposable absorbent article with a graphic disposed thereon that facilitates or promotes discretionary use of said article at least in part because the graphic is adapted to blend in with, be the same or similar to, be coordinated with, or have a common theme with a second graphic disposed on a second article that will be proximate to said disposable absorbent article during use of the disposable absorbent article; a plurality of stock-keeping units (SKUs) (or other designator of a product or products within a given product line), each corresponding to a disposable absorbent article having a different graphic disposed thereon, so that users or caregivers may select an article bearing a graphic that will blend with, or be the same as, or similar to, or coordinate with the graphic(s) on other articles proximate to said disposable absorbent article during use of the disposable absorbent article; kits that include one or more disposable absorbent articles and at least one second article of manufacture adapted to be used with said disposable absorbent article, wherein both the disposable absorbent article and the second article of manufacture have the same or similar graphics, or graphics relating to a common theme, or coordinated graphics, such that use of the disposable absorbent article together with the second article of manufacture promotes discretionary use of said disposable absorbent article; and/or marketing methods and/or tangible media that make a user, caregiver, or other potential consumer aware of the ability to promote discretionary use of a disposable absorbent article having a graphic disposed thereon by selecting a disposable absorbent article having a graphic that is adapted to blend in with, be the same or similar to, or have a common theme as, or be coordinated with a second graphic on a second article that will be proximate to said disposable absorbent article during use of the disposable absorbent article.

### SUMMARY

We have determined that discretionary use of disposable absorbent articles may be promoted in a variety of different contexts—e.g., when the disposable absorbent article is proximate to: an undergarment (as with a disposable absorbent liner), a chair or furniture (as with a disposable absorbent pad), a bed (as with a disposable absorbent bed pad), a sleeping bag (as with a disposable absorbent liner or pad for the sleeping bag), and the like—by providing disposable absorbent articles having a graphic disposed thereon that is adapted to blend with, be the same or similar to, be coordinated with, or have a common theme with a second graphic disposed on a second article of manufacture that will be proximate or adjacent to said disposable absorbent article when it is used (e.g., an undergarment, a bed, a chair or other furniture, a sleeping bag, or the like, that will be proximate to the disposable absorbent article during use of the disposable absorbent article).

In one representative embodiment of the present invention, a disposable absorbent article comprises a graphic disposed thereon that is adapted to blend in with, be the same or similar to, be coordinated with, or have a common theme with a second article of manufacture that will be proximate to said disposable absorbent article during use of the disposable absorbent article. For example, a disposable absorbent chair pad comprising a generally brown, plaid pattern disposed thereon may be used with chairs having a graphic that blends with, is the same as, or similar to, or coordinated with, this generally brown, plaid pattern. Alternatively, a disposable absorbent liner or sheet having a green color might be used with a sleeping bag having a green interior. Or a disposable absorbent article (e.g., a disposable, absorbent, boxer-brief-style article) having a graphic disposed thereon, such as black pin stripes on a blue background, may be used with woven lounge pants having the same or a similar pattern.

In another representative embodiment of the present invention, a specific type of disposable absorbent article, e.g. a disposable absorbent sheet or liner, is provided such that the disposable absorbent article is available in at least two different versions, each version having a different graphic disposed thereon (e.g., one stock keeping unit, or SKU, corresponding to a disposable absorbent sheet comprising a generally brown plaid pattern; and a second stock keeping unit, or SKU, corresponding to a plain, green color). In effect, a product line of the disposable absorbent article, such as a disposable absorbent liner or sheet, is available with different graphics so that a user, caregiver, or consumer can select that disposable absorbent article bearing a graphic that will help promote discretionary use of said disposable absorbent article (e.g., by selecting a disposable absorbent article having a graphic that will blend in with, be the same as or similar to, be coordinated with, or have a common theme with a second article of manufacture that will be in proximity to the disposable absorbent article during use of the disposable absorbent article).

In another representative embodiment of the present invention, a message embodied in a tangible medium, and adapted to be communicated to a user, caregiver, or consumer, associates use of a disposable absorbent article comprising a graphic disposed thereon with discretionary use of the disposable absorbent article. So, for example, a package, such as a plastic bag, may have a message disposed thereon in the form of a statement associating discreet use of the disposable absorbent articles contained therein by virtue of a graphic adapted to blend in with, be the same or similar to, be coordinated with, or have a common theme with a second article of manufacture that will be proximate to said disposable absorbent article during use thereof.

Another version of the present invention is a kit comprising a disposable absorbent article comprising a graphic disposed thereon and a second article of manufacture having a second graphic disposed thereon, wherein the graphic and second graphic are the same, similar, coordinated, or part of a common theme such that use of the disposable absorbent article with the second article of manufacture helps facilitate discretionary use of the disposable absorbent article (e.g., a disposable, absorbent boxer-brief with a denim appearance and woven lounge pants having a denim appearance).

In another version of the invention, the number of different disposable absorbent articles in a product line, each having a different graphic disposed thereon and adapted to blend in with, match, be the same as or similar to, be coordinated with, or have a common theme with a second graphic disposed on a second article of manufacture, such as a sleeping bag, is reduced by ascertaining the second graphic present on the majority of products corresponding to the second article of manufacture. For example, if the majority of woven sleeping bags have an interior surface with a specific color of green, then the disposable absorbent article, such as a disposable liner or pad, could be made with the same color of green—and not be made available in other patterns or colors. In this way the needs of a majority of potential users of the liner are met while reducing the complexity and cost of the product line. One version of this approach would involve collaborating with one or more manufacturers of the second article of manufacture, in this case a sleeping bag, to more precisely determine the graphics disposed on said second articles of manufacture, thereby facilitating a closer match between the graphic disposed on the disposable absorbent article and the second graphic disposed on the second article of manufacture.

In another version of this invention, a manufacturer of the disposable absorbent article having a graphic disposed thereon and adapted to facilitate discreet use of said article when used in proximity to a second article of manufacture may co-promote, co-brand, or conduct other marketing activities with the manufacturer of the second article of manufacture.

In another version of the invention, a component or sub-assembly of a disposable absorbent article, such as a liquid-impermeable layer, comprises a graphic disposed thereon adapted to blend in with, match, be the same as or similar to, be coordinated with, or have a common theme with a second graphic disposed on a second article of manufacture. The component or sub-assembly may be made by the manufacturer of the disposable absorbent article that will employ the component or sub-assembly. Alternatively, the component or sub-assembly may be made by a supplier to the manufacturer of the disposable absorbent article, and then sold to the manufacturer for use in making the corresponding disposable absorbent article that will employ the component or sub-assembly.

In some versions of the invention, the component is a decal, film, or other such material on which is disposed a graphic adapted to blend in with, match, be the same as or similar to, be coordinated with, or have a common theme with a second graphic disposed on a second article of manufacture. For example, a decal or film also comprising an adhesive may be used to attach the decal or film to other parts of a disposable absorbent particle. If the decal or film is liquid impermeable, and is being attached to a liquid-permeable layer, then the decal or film may be perforated or have openings so that the liquid-transport properties of the liquid-permeable layer are not significantly diminished (e.g., such that liquid pools on the surface of the disposable absorbent article rather than be transported through the liquid-permeable layer to, for example, an absorbent core made of cellulosic fiber).

These and other versions, embodiments, and examples of the invention are discussed elsewhere in this application.

### DRAWINGS

FIG. 1 representatively illustrates a perspective view of an example of a disposable absorbent article that can employ a graphic adapted to promote discretionary use of said article.

FIG. 2 representatively shows a plan view of the disposable absorbent article of FIG. 1 in an unfastened, stretched, and laid-flat condition with the surface of the article which contacts the wearer's skin facing the viewer and with portions of the article partially cut away to show the underlying features;

FIG. 3 representatively shows a perspective view of another example of a disposable absorbent article (a diaper pant) that can employ a graphic adapted to discretionary use of said article.

FIG. 4 representatively shows a plan view of the disposable absorbent article of FIG. 3 in an unfastened, stretched and laid flat condition with the surface of the article which contacts the wearer's skin facing the viewer and with portions of the article partially cut away to show the underlying features.

FIGS. 5A, 5B, 5C, and 5D representatively show one embodiment of a disposable absorbent article having a graphic disposed thereon (in this case a disposable absorbent pad) and a second article of manufacture having a second graphic disposed thereon (in this case a chair) proximate to said disposable absorbent article.

FIGS. 6A, 6B, 6C, and 6D representatively show one embodiment of a disposable absorbent article having a graphic disposed thereon (in this case a disposable absorbent pad or liner) and a second article of manufacture having a second graphic disposed thereon (in this case a sleeping bag) proximate to said disposable absorbent article.

FIGS. 7A and 7B representatively show one embodiment of a disposable absorbent article having a graphic disposed thereon.

Corresponding reference characters indicate corresponding parts throughout the drawings.

### Definitions

Within the context of this specification, each term or phrase below includes the following meaning or meanings:

“Attach” and its derivatives refer to the joining, adhering, connecting, bonding, sewing together, or the like, of two elements. Two elements will be considered to be attached together when they are integral with one another or attached directly to one another or indirectly to one another, such as when each is directly attached to intermediate elements. “Attach” and its derivatives include permanent, releasable, or refastenable attachment. In addition, the attachment can be completed either during the manufacturing process or by the end user.

“Bond” and its derivatives refer to the joining, adhering, connecting, attaching, sewing together, or the like, of two elements. Two elements will be considered to be bonded together when they are bonded directly to one another or indirectly to one another, such as when each is directly bonded to intermediate elements. “Bond” and its derivatives include permanent, releasable, or refastenable bonding.

“Coform” refers to a blend of meltblown fibers and absorbent fibers such as cellulosic fibers that can be formed by air forming a meltblown polymer material while simultaneously blowing air-suspended fibers into the stream of meltblown fibers. The coform material may also include other materials, such as superabsorbent materials. The meltblown fibers and absorbent fibers are collected on a forming surface, such as provided by a foraminous belt. The forming surface may include a gas-pervious material that has been placed onto the forming surface.

“Connect” and its derivatives refer to the joining, adhering, bonding, attaching, sewing together, or the like, of two elements. Two elements will be considered to be connected together when they are connected directly to one another or indirectly to one another, such as when each is directly connected to intermediate elements. “Connect” and its derivatives include permanent, releasable, or refastenable connection. In addition, the connecting can be completed either during the manufacturing process or by the end user.

“Disposable” refers to articles which are designed to be discarded after a limited use rather than being laundered or otherwise restored for reuse.

The terms “disposed on,” “disposed along,” “disposed with,” or “disposed toward” and variations thereof are intended to mean that one element can be integral with another element, or that one element can be a separate structure bonded to or placed with or placed near another element.

“Elastic,” “elasticized,” “elasticity,” and “elastomeric” mean that property of a material or composite by virtue of which it tends to recover its original size and shape after removal of a force causing a deformation. Suitably, an elastic material or composite can be elongated by at least 25 percent (to 125 percent) of its relaxed length and will recover, upon release of the applied force, at least 40 percent of its elongation.

“Extensible” refers to a material or composite which is capable of extension or deformation without breaking, but does not substantially recover its original size and shape after removal of a force causing the extension or deformation. Suitably, an extensible material or composite can be elongated by at least 25 percent (to 125 percent) of its relaxed length.

“Fiber” refers to a continuous or discontinuous member having a high ratio of length to diameter or width. Thus, a fiber may be a filament, a thread, a strand, a yarn, or any other member or combination of these members.

“Fluid” refers to urine, a bowel movement (“BM”), a urine simulant, a BM simulant, or other such liquid or material.

“Hydrophilic” describes fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90 degrees are designated “wettable” or hydrophilic, and fibers having contact angles greater than 90 degrees are designated “nonwettable” or hydrophobic.

“Layer” when used in the singular can have the dual meaning of a single element or a plurality of elements.

“Liquid impermeable,” when used in describing a layer or multi-layer laminate means that liquid, such as urine, will not pass through the layer or laminate, under ordinary use conditions, in a direction generally perpendicular to the plane of the layer or laminate at the point of liquid contact.

“Liquid permeable” refers to any material that is not liquid impermeable.

“Meltblown” refers to fibers formed by extruding a molten thermoplastic material through a plurality of fine, usually circular, die capillaries as molten threads or filaments into converging high velocity gas (e.g., air) streams, generally heated, which attenuate the filaments of molten thermoplastic material to reduce their diameters. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblowing processes can be used to make fibers of various dimensions, including macrofibers (with average diameters from about 40 to about 100 microns), textile-type fibers (with average diameters between about 10 and 40 microns), and microfibers (with average diameters less than about 10 microns). Meltblowing processes are particularly suited to making microfibers, including ultra-fine microfibers (with an average diameter of about 3 microns or less). A description of an exemplary process of making ultra-fine microfibers may be found in, for example, U.S. Pat. No. 5,213,881 to Timmons, et al. Meltblown fibers may be continuous or discontinuous and are generally self-bonding when deposited onto a collecting surface.

“Member” when used in the singular can have the dual meaning of a single element or a plurality of elements.

“Nonwoven” and “nonwoven web” refer to materials and webs of material that are formed without the aid of a textile weaving or knitting process. For example, nonwoven materials, fabrics or webs have been formed from many processes such as, for example, meltblowing processes, spunbonding processes, air laying processes, and bonded carded web processes.

“Stretchable” means that a material can be stretched, without breaking, by at least 25 percent (to 125 percent of its initial (unstretched) length) in at least one direction. Elastic materials and extensible materials are each stretchable materials.

“Superabsorbent material” refers to a water-swellable, water-insoluble organic or inorganic material capable, under the most favorable conditions, of absorbing at least about ten times its weight and, more desirably, at least about thirty times its weight in an aqueous solution containing about 0.9 weight percent sodium chloride.

These terms may be defined with additional language in the remaining portions of the specification.

### Description

### Representative Disposable Articles and Materials and Sub-Assemblies Employed Therein

A variety of disposable absorbent articles may employ a graphic disposed thereon adapted to promote discreet use and/or the dignity of the wearer or user by hiding, blending, camouflaging, or matching the article when used with a second graphic disposed on a second article of manufacture proximate to said disposable absorbent article during use of the disposable absorbent article. Such disposable absorbent articles include: disposable absorbent diapers, disposable absorbent training pants, disposable absorbent overnight garments, disposable absorbent liners (e.g., for use by women during light menstruation or for men or women for light incontinence problems); disposable absorbent garments for adult incontinence problems; disposable absorbent feminine-care pads (with or without wings or tabs); disposable absorbent pads or liners for use with chairs, beds, sleeping bags), etc; and the like.

The paragraphs that follow describe a few representative examples of the kinds of disposable absorbent articles that can employ a graphic adapted to facilitate discretionary use of said articles. Generally such articles comprise a liquid-impermeable layer, which in the following paragraphs is called an “outer cover”; a liquid-permeable layer, which in the following paragraphs is called a “body-side liner”; and an optional absorbent core. Most disposable absorbent articles have these basic elements, with the absorbent core positioned between the liquid-permeable layer and the liquid-impermeable layer, which are attached to one another (of course other elements may be present, as is clear in the description given below). In the paragraphs that follow, examples of the kinds of materials from which these basic elements may be made are described. While the representative examples described in the following paragraphs are disposable absorbent articles to be worn, the present invention encompasses other types of disposable absorbent articles, such as an absorbent liner for a bed or chair, a liner for a sleeping bag, a pad or liner for an undergarment, and the like. The same kinds of materials described below for use in making disposable absorbent articles that are worn may be used when making disposable absorbent articles like that described in the preceding sentence.

As is described in more detail below, the graphic adapted to facilitate discretionary use of a disposable absorbent article will generally be disposed on a surface or layer that may be seen by other people. So, for example, for a disposable absorbent chair pad, a graphic may be disposed on a liquid-permeable layer that will be oriented away from the chair surface so that the graphic is visible to others. The liquid-permeable layer with a graphic disposed thereon will be attached to a liquid-impermeable layer oriented toward the chair surface, thereby serving to protect the chair from liquid. An absorbent core may be sandwiched between the two layers, thereby helping to absorb and hold any liquid passing through the liquid-permeable layer into the absorbent core. The graphic disposed on the disposable absorbent chair pad will be selected so that it is the same or similar as, blends with, is camouflaged by, coordinates with, or has a similar theme with a second graphic disposed on the chair itself. In other words, the relationship between the graphic on the disposable absorbent article and the second graphic on a second article of manufacture proximate to said disposable absorbent article during use thereof is such that the disposable absorbent article is not readily detectable, or is less readily detectable, visually, by an observer (relative to, for example, a conventional disposable article which is generally white in color). Additional detail on the visual coordination of an absorbent product with a package is disclosed in U.S. patent application Ser. No. 11/025,645, entitled “Visually Coordinated Absorbent Product” to Theresa M. Zander et al.; and U.S. patent application Ser. No. 10/749,871, also entitled “Visually Coordinated Absorbent Product” to Theresa M. Zander et al. Both of these U.S. applications are hereby incorporated by reference in a manner consistent herewith.

For disposable absorbent articles that are worn next to the body (as opposed to disposable absorbent articles that are placed next to second articles of manufactures such as chairs, other kinds of furniture, beds, sleeping bags, floors, etc.), the graphic will generally be disposed on a liquid-impermeable layer or other component that is visible, partly visible, or potentially visible to people other than the user of the disposable absorbent article when the disposable absorbent article is used.

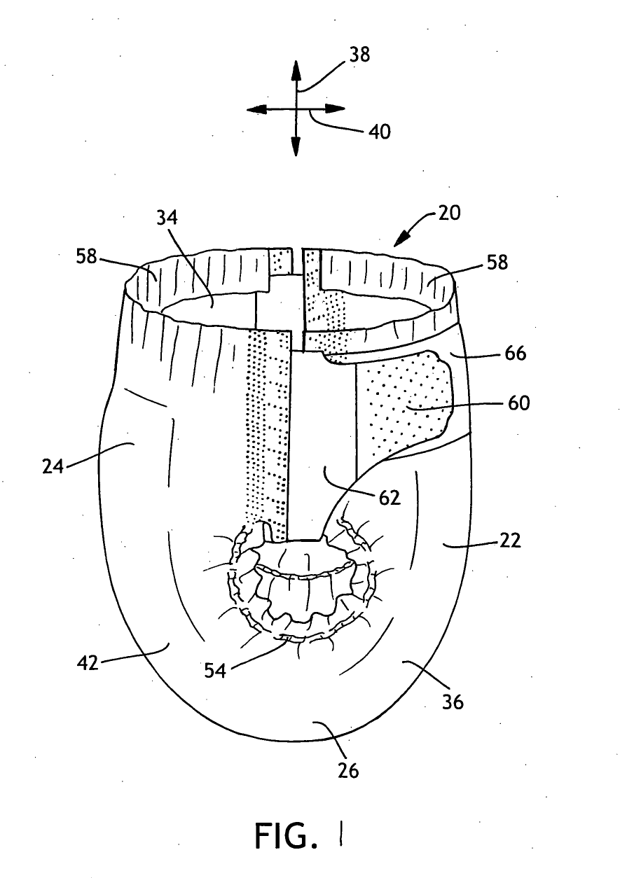

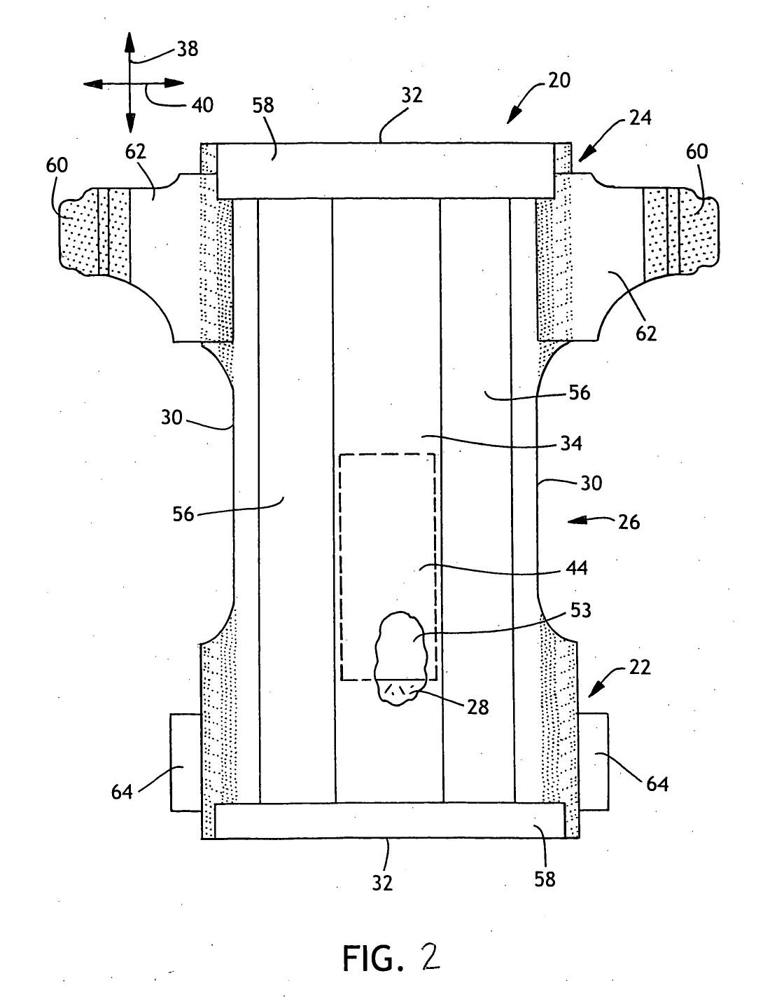

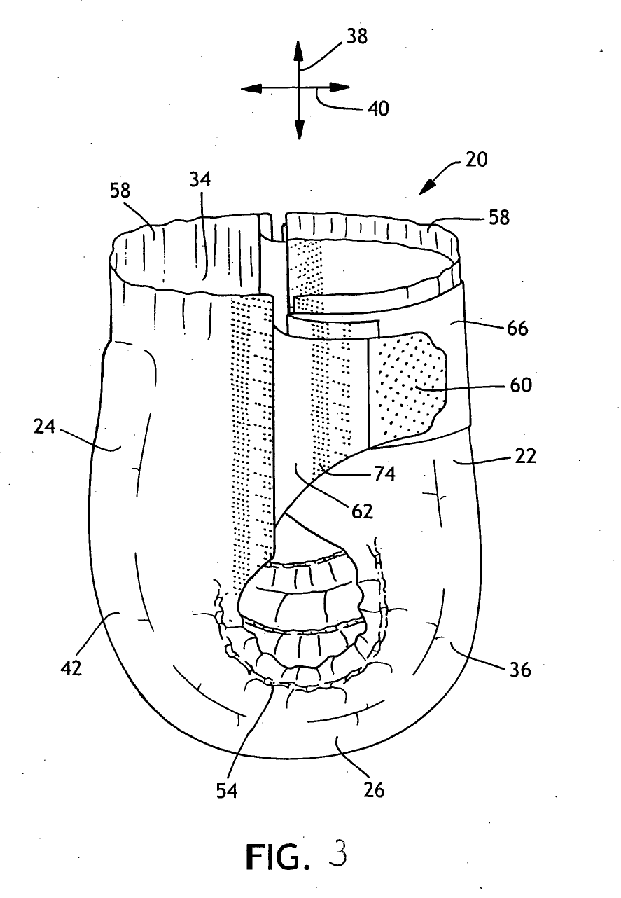

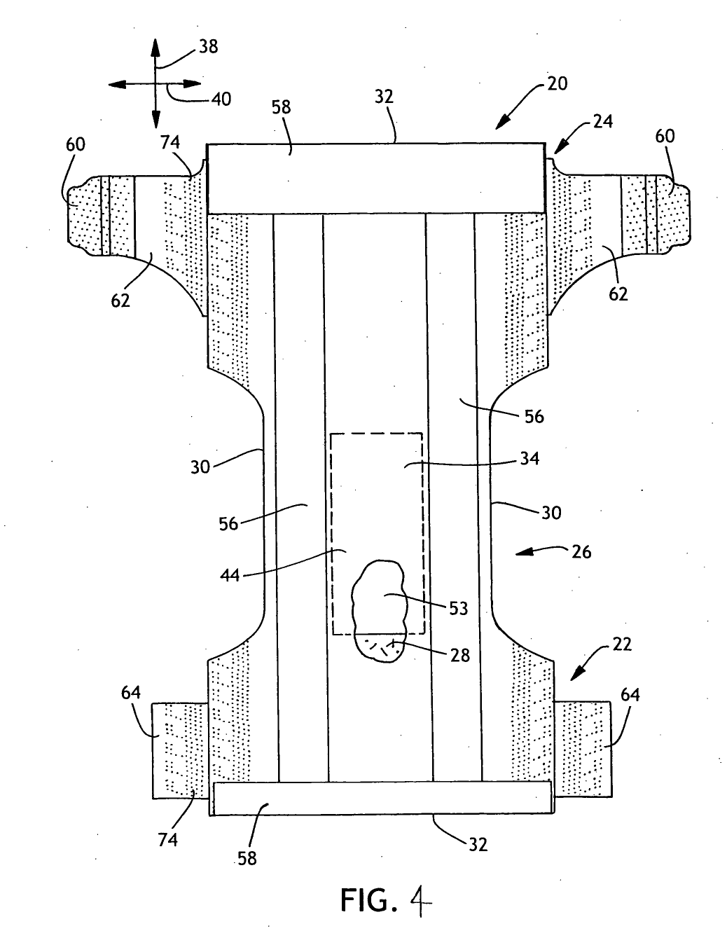

FIG. 1 representatively illustrates an example of a refastenable disposable diaper, as generally indicated at 20, which employs a graphic adapted to facilitate discretionary use of said article. FIG. 2 representatively illustrates the refastenable diaper of FIG. 1 in an unfastened, stretched and laid flat configuration with the surface of the diaper adapted to contact the wearer's skin facing the viewer and with portions of the diaper partially cut away to show the underlying features. FIG. 3 representatively illustrates another example of an absorbent article that can employ a graphic adapted to facilitate discretionary use of said article, a diaper pant generally indicated at 20. FIG. 4 representatively illustrates the prefastened diaper pant of FIG. 3 in an unfastened, stretched and laid flat configuration with the surface of the diaper pant adapted to contact the wearer's skin facing the viewer and with portions of the diaper pant partially cut away to show the underlying features. As illustrated in FIG. 2 and FIG. 4, the diaper/diaper pant 20 defines a front waist region 22, a back waist region 24, a crotch region 26 that extends between and connects the front and back waist regions 22 and 24, a longitudinal direction 38 and a lateral direction 40. The front waist region 22 includes the portion of the diaper/diaper pant 20 that, when worn, is positioned on the front of the wearer while the back waist region 24 includes the portion of the diaper/diaper pant 20 that, when worn, is positioned on the back of the wearer. The crotch region 26 of the diaper/diaper pant 20 includes the portion of the diaper/diaper pant 20 that, when worn, is positioned between the legs of the wearer and covers the lower torso of the wearer.

The diaper/diaper pant 20 defines a pair of laterally opposed side edges 30, a pair of longitudinally opposed waist edges 32, an interior surface 34 that is configured to contact the wearer, and an exterior surface 36 opposite the interior surface 34 that is configured to contact the wearer's clothing in use. The illustrated diaper/diaper pant 20 also includes a substantially liquid impermeable outer cover 42 and a liquid permeable bodyside liner 44 that can be connected to the outer cover 42 in a superposed relation. An absorbent core 28 is located between the outer cover 42 and the bodyside liner 44. The laterally opposed side edges 30 of the diaper/diaper pant 20 are generally defined by the side edges of the outer cover 42 that further define leg openings that may be curvilinear. The waist edges 32 of the diaper/diaper pant 20 are generally defined by the waist edges of the outer cover 42 and define a waist opening that is configured to encircle the waist of the wearer when worn. The absorbent core 28 is configured to contain and/or absorb body exudates discharged from the wearer. The diaper/diaper pant 20 may further include leg elastics 54, containment flaps 56 and waist elastics 58 as are known to those skilled in the art. It should be recognized that individual components of the diaper/diaper pant 20 may be optional depending upon the intended use of the diaper/diaper pant 20.

The diaper/diaper pant 20 may further include refastenable mechanical fasteners 60. The mechanical fasteners 60 releasably engage the opposed side edges 30 of the diaper/diaper pant 20 in the opposite waist regions. The mechanical fasteners 60 can include a variety of materials and surfaces known for mechanical engagement such as buttons, pins, snaps, adhesive tape fasteners, cohesives, mushroom-and-loop fasteners, and hook and loop fasteners. Further, the disposable diaper/diaper pant 20 may include an attachment panel 66 located on the front or back waist region 22 and 24, opposite the fasteners 60 to which the fasteners 60 can be releasably engaged during use of the diaper/diaper pant 20.

The diaper/diaper pant 20 may be of various suitable shapes. For example, in the unfastened configurations as illustrated in FIG. 2 and FIG. 4, the diaper/diaper pant 20 may have an overall rectangular shape, T-shape or an approximately hourglass shape. In the shown embodiments, the diaper/diaper pant 20 has a generally I-shape in an unfastened configuration.

The various components of the diaper/diaper pant 20 are integrally assembled together employing various types of suitable attachment means, such as adhesive, sonic, and thermal bonds, or combinations thereof. In the shown embodiments, for example, the outer cover 42 and bodyside liner 44 are assembled to each other and to the absorbent core 28 with adhesive, such as a hot melt, pressure-sensitive adhesive. The adhesive may be applied as a uniform continuous layer of adhesive, a patterned layer of adhesive, a sprayed pattern of adhesive, or an array of separate lines, swirls or dots of adhesive. Alternatively, the absorbent core 28 may be connected to the outer cover 42 using conventional fasteners such as buttons, hook and loop type fasteners, adhesive tape fasteners, and the like. The other components of the diaper/diaper pant 20 may be suitably connected together using similar means. Similarly, other diaper components, such as the elastic members 54 and 58 and the mechanical fasteners 60, may be assembled into the diaper/diaper pant 20 article by employing the above-identified attachment mechanisms. Desirably, the majority of the diaper components are assembled together using ultrasonic bonding techniques for reduced manufacturing cost.

The outer cover 42 of the diaper/diaper pant 20, as representatively illustrated in FIGS. 1 and 3, may suitably be composed of a material which is either liquid permeable or liquid impermeable. It is generally preferred that the outer cover 42 be formed from a material that is substantially impermeable to liquids. A typical outer cover 42 can be manufactured from a thin plastic film or other flexible liquid-impermeable material. For example, the outer cover 42 may be formed from a polyethylene film having a thickness of from about 0.013 millimeter (0.5 mil) to about 0.051 millimeter (2.0 mils). The materials of the outer cover 42 can be thermally or adhesively laminated together. Suitable laminate adhesives, which can be applied continuously or intermittently as beads, a spray, parallel swirls, or the like, can be obtained from Bostik-Findley, Inc., of Wauwatosa, Wis., U.S.A., or from National Starch and Chemical Company, Bridgewater, N.J., U.S.A. If it is desired to present the outer cover 42 with a more cloth-like feeling, the outer cover 42 may be formed from a polyolefin film having a nonwoven web laminated to the exterior surface thereof, such as a spunbond web of polyolefin fibers. For example, a stretch-thinned polypropylene film having a thickness of about 0.015 millimeter (0.6 mil) may be thermally laminated thereto a spunbond web of polypropylene fibers. The polypropylene fibers may have a fiber diameter of about 15 to 20 microns, which nonwoven web has a basis weight of about 17 grams per square meter (0.5 ounce per square yard). The outer cover 42 may include bicomponent fibers such as polyethylene/polypropylene bicomponent fibers. Methods of forming such cloth-like outer covers are known to those skilled in the art. The outer cover 42 may also be an extensible outer cover such as the outer covers described in U.S. Pat. No. 6,552,245 issued on Apr. 22, 2003 to Roessler et al. The outer cover 42 may also be a biaxially stretchable outer cover such as the outer covers described in U.S. patent application Ser. No. 09/698,517 filed on Oct. 27, 2000 by Vukos et al.

The outer cover 42 may be formed of a woven or nonwoven fibrous web layer which has been totally or partially constructed or treated to impart a desired level of liquid impermeability to selected regions that are adjacent or proximate the absorbent core 28. Still further, the outer cover 42 may optionally be composed of a micro-porous “breathable” material which permits vapors to escape from the absorbent core 28 while still preventing liquid exudates from passing through the outer cover 42. For example, the outer cover 42 may include a vapor permeable non-woven facing layer laminated to a micro-porous film. Suitable “breathable” outer cover materials are described in U.S. Pat. No. 5,695,868 issued Dec. 9, 1997 to McCormack et al. and U.S. Pat. No. 5,843,056 issued Dec. 1, 1998 to Good et al., the descriptions of which are hereby incorporated by reference. Still further, the outer cover 42 may also be an elastomeric material such as a stretch-thermal laminate (STL), neck-bonded laminate (NBL), or stretch-bonded laminate (SBL) material. Methods of making such materials are well known to those skilled in the art and are described in U.S. Pat. No. 4,663,220 issued May 5, 1987 to Wisneski et al., U.S. Pat. No. 5,226,992 issued Jul. 13, 1993 to Morman, and European Patent Application No. EP 0 217 032 published on Apr. 8, 1987 in the name of Taylor et al., the disclosures of which are hereby incorporated by reference. The outer cover 42 can also be embossed or otherwise provided with a matte finish to provide a more aesthetically pleasing appearance.

In order to reduce the perception that the outer cover 42 feels damp or clammy, the diapers/diaper pants 20 may include a spacer or ventilation layer (not shown in Figures) between the garment-facing surface of the absorbent core 28 and the outer cover 42. The ventilation layer may include one or more nonwoven materials, for example a spunbond-meltblown-spunbond nonwoven material.

The representative absorbent articles include a bodyside liner 44 in superimposed relation to the outer cover 42. The bodyside liner 44, as representatively illustrated in FIG. 2 and FIG. 4, suitably presents a bodyfacing surface that is compliant, soft feeling, and nonirritating to the wearer's skin. Further, the bodyside liner 44 may be less hydrophilic than the absorbent core 28, to present a relatively dry surface to the wearer, and may be sufficiently porous to be liquid permeable, permitting liquid to readily penetrate through its thickness. A suitable bodyside liner 44 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers. The bodyside liner 44 is suitably employed to help isolate the wearer's skin from liquids held in the absorbent 28. The bodyside liner 44 can also be made from extensible materials as are described in U.S. Pat. No. 6,552,245 issued on Apr. 22, 2003 to Roessler et al. The bodyside liner 44 can also be made from biaxially stretchable materials as are described in U.S. patent application Ser. No. 09/698,517 filed on Oct. 27, 2000 by Vukos et al.

Various woven and nonwoven fabrics can be used for the bodyside liner 44. For example, the bodyside liner may be composed of a meltblown or spunbond web of polyolefin fibers. The bodyside liner 44 may also be a bonded-carded web composed of natural and/or synthetic fibers. The bodyside liner 44 may be composed of a substantially hydrophobic material, and the hydrophobic material may optionally be treated with a surfactant or otherwise processed to impart a desired level of wettability and hydrophilicity. In a particular embodiment, the bodyside liner 44 is made from a nonwoven, spunbond, polypropylene fabric composed of fibers having a fiber diameter of about 21 to 23 microns formed into a web having a basis weight of about 20 grams per square meter and a density of about 0.13 grams per cubic centimeter. The fabric may be surface treated with about 0.3 weight percent of a surfactant, such as a surfactant commercially available from Hodgson Textile Chemicals, Inc. under the trade designation AHCOVEL Base N-62. The surfactant may be applied by any conventional means, such as spraying, printing, brush coating or similar techniques. The surfactant may be applied to the entire bodyside liner 44 or may be selectively applied to particular sections of the bodyside liner 44, such as the medial section along the longitudinal centerline of the diaper, to provide greater wettability of such sections. The bodyside liner 44 may further include a lotion or treatment applied thereto that is configured to be transferred to the wearer's skin. Suitable compositions for application to the bodyside liner 44 are described in U.S. Pat. No. 6,149,934 that issued to Krzysik et al. on Nov. 21, 2000.

The representative absorbent articles can include an absorbent core 28 disposed between the outer cover 42 and the bodyside liner 44. The absorbent core 28 of the diaper/diaper pant 20, as representatively illustrated in FIG. 1 and FIG. 3, may suitably include a matrix of hydrophilic fibers, such as a web of cellulosic fluff, mixed with particles of a high-absorbency material commonly known as superabsorbent material. In a particular aspect, the absorbent core 28 includes a matrix of cellulosic fluff, such as wood pulp fluff, and superabsorbent hydrogel-forming particles. The wood pulp fluff may be exchanged with synthetic, polymeric, meltblown fibers or with a combination of meltblown fibers and natural fibers. The superabsorbent particles may be substantially homogeneously mixed with the hydrophilic fibers or may be nonuniformly mixed. Alternatively, the absorbent core 28 may include a laminate of fibrous webs and superabsorbent material or other suitable matrix for maintaining a superabsorbent material in a localized area.

The absorbent core 28 may have any of a number of shapes. For example, the absorbent core 28 may be rectangular, I-shaped, or T-shaped. It is generally preferred that the absorbent core 28 is narrower in the intermediate section than in the front or rear waist sections of the diaper 20. The absorbent core 28 may be provided by a single layer or, in the alternative, may be provided by multiple layers, all of which need not extend the entire length and width of the absorbent core 28. In a particular aspect, the absorbent core 28 can be generally T-shaped with the laterally extending cross-bar of the “T” generally corresponding to the front waist region 22 of the absorbent article for improved performance.

The size and the absorbent capacity of absorbent core 28 should be compatible with the size of the intended wearer and the liquid loading imparted by the intended use of the absorbent article. Further, the size and the absorbent capacity of the absorbent core 28 can be varied to accommodate wearers ranging from infants through adults. In addition, it has been found that the densities and/or basis weights of the absorbent core 28 can be varied.

The high-absorbency material may be selected from natural, synthetic, and modified natural polymers and materials. The high-absorbency materials may be inorganic materials, such as silica gels, or organic compounds, such as crosslinked polymers. The term “crosslinked” refers to methods for effectively rendering normally water-soluble materials substantially water insoluble but swellable. Such methods include, for example, physical entanglement, crystalline domains, covalent bonds, ionic complexes and associations, hydrophilic associations such as hydrogen bonding, and hydrophobic associations or Van der Waals forces.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinyl morpholinone), poly(vinyl alcohol), and mixtures and copolymers thereof. Further polymers suitable for use in the absorbent core 28 include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthan gum, locust bean gum, and the like. Mixtures of natural and wholly or partially synthetic absorbent polymers can also be useful.

The high absorbency material may be in any of a wide variety of geometric forms. As a general rule, it is preferred that the high absorbency material be in the form of discreet particles. However, the high absorbency material may also be in the form of fibers, flakes, rods, spheres, needles, or the like. In general, the high absorbency material is present in the absorbent core 28 in an amount of from about 5 to about 90 percent by weight, desirably in an amount of at least about 30 percent by weight, and even more desirably in an amount of at least about 50 percent by weight based on a total weight of the absorbent core 28. For example, in a particular aspect, the absorbent core 28 may include a laminate which includes at least about 50 percent by weight and desirably at least about 70 percent by weight of high-absorbency material overwrapped by a fibrous web or other suitable material for maintaining the high-absorbency material in a localized area.

An example of high-absorbency material suitable for use is DRYTECH 2035 polymer available from Dow Chemical, a business having offices in Midland, Mich. Other suitable superabsorbents may include FAVOR SXM 880 polymer obtained from Stockhausen, a business having offices in Greensboro, N.C.

Optionally, a substantially hydrophilic tissue or nonwoven wrapsheet (not illustrated) may be employed to help maintain the integrity of the structure of the absorbent core 28. The wrapsheet is typically placed about the absorbent core 28 over at least the two major facing surfaces thereof. The wrapsheet may be composed of an absorbent cellulosic material, such as creped wadding or a high wet-strength tissue. In one aspect, the wrapsheet may be configured to provide a wicking layer that helps to rapidly distribute liquid over the mass of absorbent fibers constituting the absorbent core 28.

Due to the thinness of absorbent core 28 and the high absorbency material within the absorbent core 28, the liquid uptake rates of the absorbent core 28, by itself, may be too low, or may not be adequately sustained over multiple insults of liquid into the absorbent core 28. To improve the overall liquid uptake and air exchange, the diaper/diaper pant 20 may further include a porous, liquid-permeable layer of surge management material 53, as representatively illustrated in FIG. 2 and FIG. 4. The surge management layer 53 is typically less hydrophilic than the absorbent core 28, and has an operable level of density and basis weight to quickly collect and temporarily hold liquid surges, to transport the liquid from its initial entrance point and to substantially completely release the liquid to other parts of the absorbent core 28. This configuration can help prevent the liquid from pooling and collecting on the portion of the diaper/diaper pant 20 positioned against the wearer's skin, thereby reducing the feeling of wetness by the wearer. The structure of the surge management layer 53 also generally enhances the air exchange within the diaper/diaper pant 20.

Various woven and nonwoven fabrics can be used to construct the surge management layer 53. For example, the surge management layer 53 may be a layer composed of a meltblown or spunbond web of synthetic fibers, such as polyolefin fibers. The surge management layer 53 may also be a bonded-carded-web or an airlaid web composed of natural and synthetic fibers. The bonded-carded-web may, for example, be a thermally bonded web that is bonded using low melt binder fibers, powder, or adhesive. The webs can optionally include a mixture of different fibers. The surge management layer 53 may be composed of a substantially hydrophobic material, and the hydrophobic material may optionally be treated with a surfactant or otherwise processed to impart a desired level of wettability and hydrophilicity. In a particular aspect, the surge management layer 53 includes a hydrophobic, nonwoven material having a basis weight of from about 30 to about 120 grams per square meter.

The absorbent articles can include additional components. For example, as representatively illustrated in FIGS. 1-4, the disposable diaper/diaper pant 20 may include a pair of containment flaps 56 that are configured to provide a barrier to the lateral flow of body exudates. The containment flaps 56 may be located along the laterally opposed side edges 30 of the diaper/diaper pant adjacent the side edges of the absorbent core 28. Each containment flap 56 typically defines an unattached edge that is configured to maintain an upright, perpendicular configuration in at least the crotch region 26 of the diaper/diaper pant 20 to form a seal against the wearer's body. The containment flaps 56 may extend longitudinally along the entire length of the absorbent core 28 or may only extend partially along the length of the absorbent core 28. When the containment flaps 56 are shorter in length than the absorbent core 28, the containment flaps 56 can be selectively positioned anywhere along the side edges 30 of diaper/diaper pant 20 in the crotch region 26. In a particular aspect, the containment flaps 56 extend along the entire length of the absorbent core 28 to better contain the body exudates. Such containment flaps 56 are generally well known to those skilled in the art.

The diaper/diaper pant 20 may further include elastics at the waist edges 32 and side edges 30 of the diaper/diaper pant 20 to further prevent leakage of body exudates and support the absorbent core 28. For example, as representatively illustrated in FIGS. 1-4, the diaper/diaper pant 20 may include a pair of leg elastic members 54 that are connected to the laterally opposed side edges 30 of the diaper/diaper pant 20 in the crotch region 26. The diaper/diaper pant 20 may also include a pair of waist elastic members 58 that is connected to the longitudinally opposed waist edges 32 of the diaper/diaper pant 20. The leg elastics 54 and waist elastics 58 are generally adapted to fit about the legs and waist of a wearer in use to maintain a positive, contacting relationship with the wearer to effectively reduce or eliminate the leakage of body exudates from the diaper/diaper pant 20.

Materials suitable for use as the leg elastics 54 and waist elastics 58 are well known to those skilled in the art. Exemplary of such materials are sheets or strands or ribbons of a polymeric, elastomeric material that may be adhered to the outer cover 42 in a stretched position, or that may be attached to the outer cover 42 while the outer cover is pleated, such that elastic constrictive forces are imparted to the outer cover 42. The leg elastics 54 may also include such materials as polyurethane, synthetic, and natural rubber. The waist elastics 58 may be formed by elastic strands attached to the outer cover 42 or they may be formed by attaching separate pieces of stretchable materials to the waist regions of the article. For example, the waist elastics 58 may include a piece of stretch-bonded laminate material attached to the interior surface 34 of the article to form a waistband. Elasticity may be added or incorporated into the waist opening of absorbent articles utilizing a variety of known approaches.

The absorbent articles may include one or more components that extend laterally outward from the longitudinal sides of the article. Typically, the longitudinal sides are defined by the materials forming the chassis of the diaper/diaper pant 20. The chassis may be defined by the outer cover 42 and bodyside liner 44 materials. Components that extend laterally outward may include front ear portions 64 and back ear portions 62. The front ear portions 64 and the back ear portions 62 may be formed of one or more materials and may include laminates of materials. The front ear portions 64 and the back ear portions 62 improve the fit of the absorbent article. More specifically, the front ear portions 64 may provide additional coverage around the waist of the wearer and they may assist caregivers with positioning the front waist region 22 on the wearer of the article. The front ear portions 64 may also include mechanical fastening materials such that the front ear portions 64 contribute to the overall fastening system of the article. The back ear portions 62 may also provide coverage around the waist of the wearer. More specifically, the back ear portions 62 may provide the bridging material between the back waist region 24 of the article and the front waist region 22 such that the back ear portions 62 form part of the article's waist opening and an upper edge of the article's leg openings. Additionally, the back ear portions 62 may include fastening materials that facilitate joining of the back waist region 24 with the front waist region 22. For example, the back ear portions 62 may include fasteners 60 selected for engagement with an attachment panel 66 in the front waist region 22 of the article.

Many presently available diapers typically include back ear portions 62 that include a stretchable material. When the back ear portions 62 include a stretchable material, the back ear portions 62 may increase the range with which the fasteners 60 may be engaged into the attachment panel 66 or directly into the outer cover 42. Further, when the back ear portions 62 include a stretchable material, the article may be worn by a greater range of users as a result of the increased fit range. An exemplary material from which the back ear portions 62 may be constructed is a necked bonded laminate material having two nonwoven (e.g. spunbond) facings with an elastomeric film (e.g. KRATON film) laminated in between. Other suitable stretchable materials are known in the art. Depending on the design of the article, it may also be desirable for the front ear portions 64 to include a stretchable material.

When the product form of the absorbent article is a training pant or a swim pant, the back ear portions 62 and the front ear portions 64 are understood to include the side panels that are attached to the longitudinal sides 30 of the article and also are attached to each other to form side seams of the article. Typically, the side panels of training pants and swim pants are made from stretchable materials. The side panels' ability to stretch allows these products to be pulled on the wearer like underpants.

The diaper pant 20 form (representatively illustrated in FIG. 2 and FIG. 3) may be described as a hybrid between an infant diaper that is typically removed and applied while the child is lying down and a training pant that is put on like underpants. The product form may be referred to as a diaper pant because the diaper pant may be applied and removed as either a diaper or a pant. A diaper pant may have a back ear portion 62 and a front ear portion 64 where the back ear portion 62 and front ear portion 64 are attached to each other by a passive side bond 74. The passive side bond 74 may be selected to be readily tearable by caregivers during the process of “converting” the diaper pant from a pant to a diaper. Desirably, the passive side bond 74 is easily opened/broken without tearing of the materials used to form the back ear portion 62 and the front ear portion 64.

With each of the product forms, the back ear portions 62 and the front ear portions 64 may be attached to the longitudinal side edges 30 of the article by bonding techniques, such as ultrasonic bonding. Use of ultrasonic bonding techniques tends to form discreet bond points. The bond patterns, as will be discussed herein, may be formed using known techniques such as adhesive, thermal, laser, and pressure that are capable of forming the patterns. Ultrasonic bonding will be referred to for purposes of example. The back ear portions 62 and the front ear portions 64 may be attached to one or more of the chassis materials using one or more bond patterns. The articles may include an attachment area 76 where the attachment area 76 includes an overlapping area of the material forming the outer cover 42 and the material forming either a back ear portion 62 or a front ear portion 64. The attachment area 76 may further include a variegated bond pattern 70 and a uniform bond pattern 72 where each bond pattern provides attachment between the outer cover 42 material and the ear portion material. The attachment area 76 may also include an overlapping area of the material forming the bodyside liner 44 and the material forming either a back ear portion 62 or a front ear portion 64. Additionally, the attachment area 76 may include an overlapping area of the material forming the outer cover 42, the material forming the bodyside liner 44 and the material forming either a back ear portion 62 or a front ear portion 64.

The variegated bond pattern 70 may be formed by a plurality of bond points that are variable in location with respect to each other. Put differently, the bond points of the variegated bond pattern 70 may not all be located equidistantly from each other. While the bond points of the variegated bond pattern 70 may be spaced irregularly with respect to each other, the variegated bond pattern 70 may have a repeating pattern appearance. The uniform bond pattern 72 may be formed by a plurality of bond points that are regular in location with respect to each other. Put differently, the bond points of the uniform bond pattern 72 are located generally equidistantly from each other. The variegated bond pattern 70 and the uniform bond pattern 72 may be immediately adjacent to each other as illustrated in FIG. 3 or the patterns may have some open space in between them. The bond patterns may be formed by ultrasonic bonds or by other bonding techniques capable of providing individual bond points.

Other examples of disposable absorbent articles that can employ a graphic adapted to facilitate discretionary use of said article include a disposable absorbent boxer or other outer-shell-type article, such as that described in U.S. Patent Publication No. 2005/0148980 published on 7 Jul. 2005, entitled “Absorbent Garment Having Outer Shell and Discreet Absorbent Assembly Adapted for Positioning Therein”; a training pant or other such disposable absorbent article, such as that described in U.S. Patent Publication No. 2006/0004341 A1 published on 5 Jan. 2006, entitled “Stretchable Absorbent Article Having Lateral and Longitudinal Stretch Properties”; and, of course, other disposable absorbent articles known to those of skill in the art.

### Representative Equipment and Processes by which a Graphic May be Associated with a Disposable Absorbent Article and/or Materials and/or Sub-Assemblies and/or Components Employed Therein

A graphic may be associated with a disposable absorbent article, including materials, sub-assemblies, and/or components thereof, using different types of equipment in a variety of ways. Flexographic printing is a conventional printing technique which uses flexible, raised rubber or photopolymer plates to carry an inked image to a substrate, such as one or more components in a disposable absorbent article, including a liquid-impermeable outer cover, a liquid-permeable liner, or other such structures. As an example, a flexographic printing apparatus is shown and/or described in U.S. Pat. No. 5,458,590 (Schleinz et al.); U.S. Pat. No. 5,566,616 (Schleinz et al.); U.S. Patent Publication No. 2003/0019374A1 (Harte); and U.S. Pat. No. 4,896,600 (Rogge et al.). Each of these patents and patent publications is hereby incorporated by reference in a manner consistent herewith. Further, a graphic may be printed, sprayed, or otherwise applied to disposable absorbent article, including materials, sub-assemblies, and/or components thereof, by any other method (e.g., ink jet, rotogravure, etc.), or a component or sub-assembly thereof.

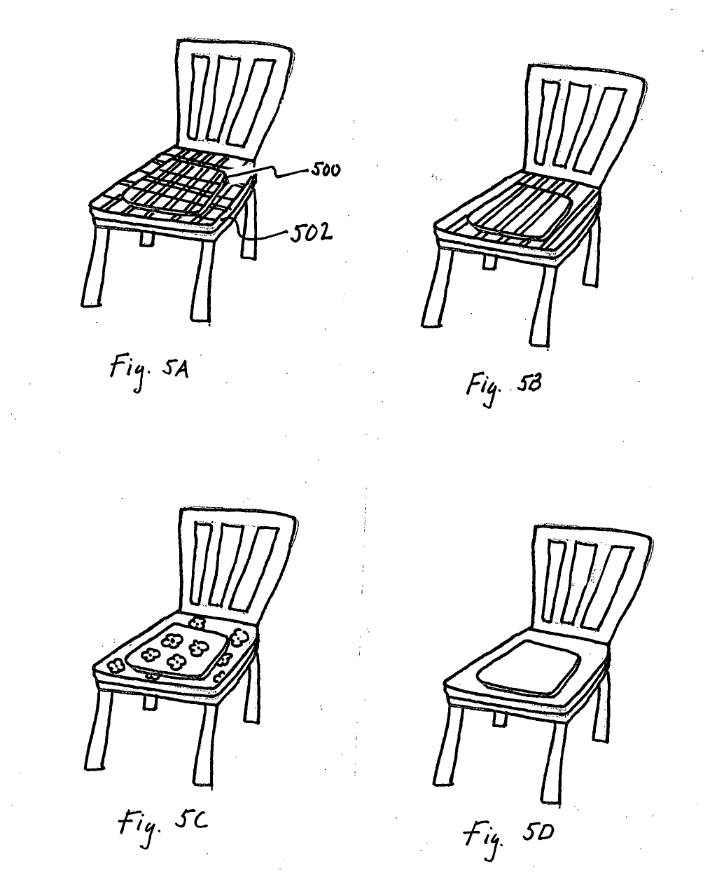

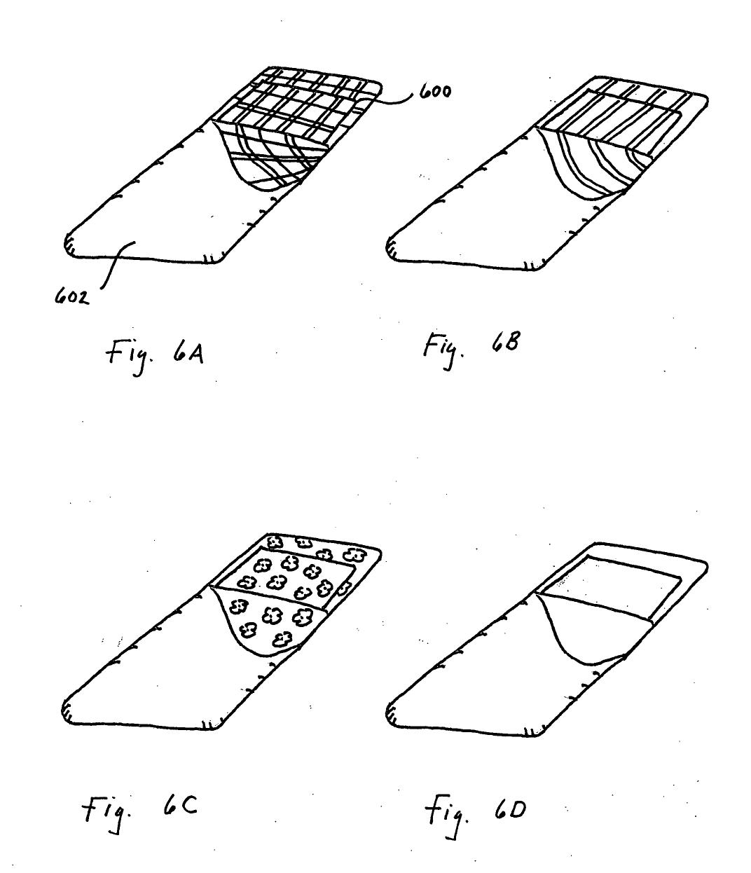

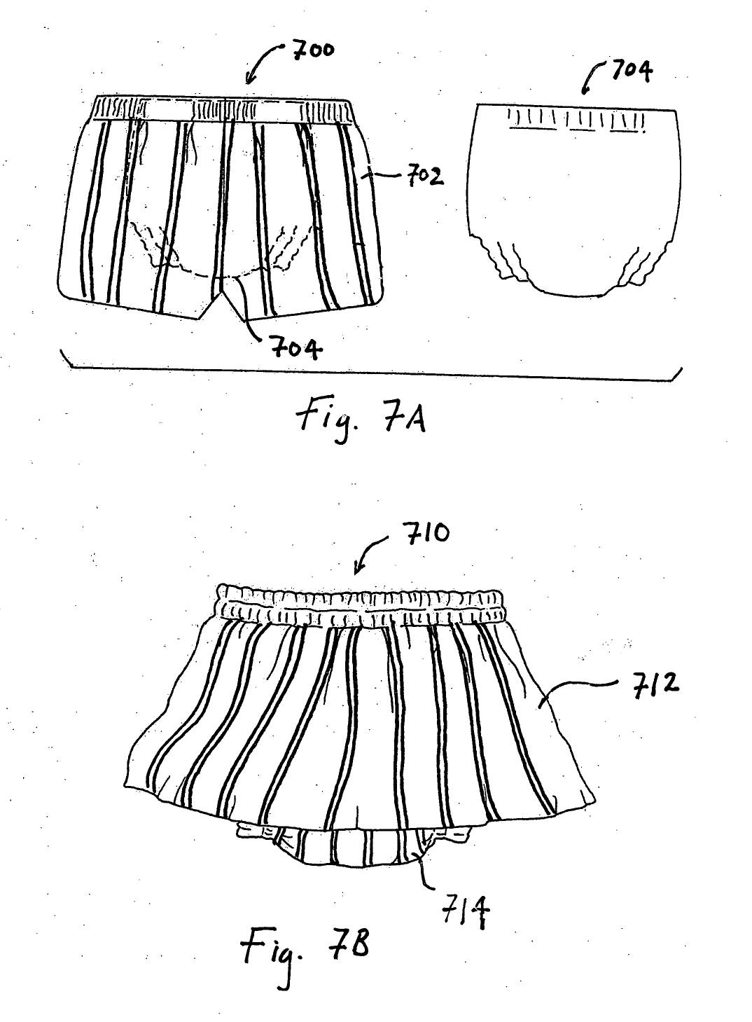

As noted elsewhere in this application, a graphic disposed on the surface of the disposable absorbent article, or a component or sub-assembly thereof (e.g., the liquid-permeable layer or liquid-impermeable layer), will be disposed on at least some portion of a surface that is visible to others, or may be visible to others (e.g., the interior of a sleeping bag may not always be visible to others). Various representative versions of the present invention are presented in FIGS. 5A through 5D (representative chair pads having a plaid graphic, a striped graphic, a floral graphic, and a graphic of a uniform color, respectively); FIGS. 6A through 6D (representative sleeping bags having a plaid graphic, a striped graphic, a floral graphic, and a graphic of a uniform color, respectively); and FIGS. 7A and 7B (a boxer brief having a striped graphic and a skirt-like article having a striped graphic).

In FIGS. 5A through 5D, a disposable absorbent pad or liner 500 is positioned on the seat of a chair 502. The surface of the pad or liner facing upward so that it will contact a user of the chair comprises a graphic, either on the surface of the pad or liner, or so that the graphic is visible to a user (e.g., the upper-most liquid-permeable layer is transparent or translucent to an underlying layer on which the graphic is disposed). In the representative version depicted in FIG. 5A, the graphic corresponds to a plaid pattern. The graphic on the disposable absorbent article is the same or similar to the second graphic disposed on the second article of manufacture with which the disposable absorbent article will be used, in this case a chair. In this representative embodiment, the color or colors [not shown] of the plaid pattern of the disposable absorbent graphic are the same as or similar to the color or colors [not shown] of the plaid pattern of the second graphic. While numerical designators are not shown in FIGS. 5B through 5D, the displayed representative embodiments correspond to the disposable absorbent article and second article of manufacture displayed in FIG. 5A. In each of the representative embodiments depicted in FIGS. 5B through 5D, the pattern and color of a graphic disposed on the depicted disposable absorbent article is the same as or similar to the pattern and color of a second graphic disposed on a second article of manufacture with which the disposable absorbent article is being employed.

In FIGS. 6A through 6D, a disposable absorbent pad or liner 600 is positioned inside a sleeping bag 602. The surface of the pad or liner that faces upward so that it will contact a user of the sleeping bag comprises a graphic, either on the surface of the pad or liner, or so that the graphic is visible to a user (e.g., the upper-most liquid-permeable layer is transparent or translucent to an underlying layer on which the graphic is disposed). In the representative version depicted in FIG. 6A, the graphic corresponds to a plaid pattern. The graphic on the disposable absorbent article is similar or identical to the second graphic disposed on the second article of manufacture with which the disposable absorbent article will be used, in this case a sleeping bag. In this representative embodiment, the color or colors [not shown] of the plaid pattern of the disposable absorbent graphic are the same as or similar to the color or colors [not shown] of the plaid pattern of the second graphic. While numerical designators are not shown in FIGS. 6B through 6D, the displayed representative embodiments correspond to the disposable absorbent article and second article of manufacture displayed in FIG. 6A. In each of the representative embodiments depicted in FIGS. 6B through 6D, the pattern and color of a graphic disposed on the depicted disposable absorbent article is the same as or similar to the pattern and color of a second graphic disposed on a second article of manufacture with which the disposable absorbent article is being employed.

In FIG. 7A, a disposable absorbent boxer-style article 700 comprises an outer shell 702 and an absorbent sub-assembly 704 (with the absorbent sub-assembly comprising an absorbent core sandwiched between a liquid-permeable layer and a liquid-impermeable layer). For ease of viewing, sub-assembly 704 is also depicted without the boxer-style outershell 702.

In FIG. 7B, a disposable absorbent skirt-like article 710 comprises an outer shell 712 and an absorbent sub-assembly 714 (with the absorbent sub-assembly comprising an absorbent core sandwiched between a liquid-permeable layer and a liquid-impermeable layer).

In the representative embodiments depicted in FIGS. 7A and 7B, the disposable absorbent articles bear a stripe-like graphic. Such articles might be worn with woven lounge-style or other such pants or garments having the same or similar graphic. Or these articles might be worn in conjunction with use of a sleeping bag having the same or similar graphic (with or without a disposable absorbent liner or pad also having the same or similar graphic).

Other embodiments and combinations are possible of course. For example, a disposable absorbent pad or liner comprising a graphic might be used with woven sheets on a bed, wherein the woven sheets comprise a second graphic that is the same or similar to the graphic on the disposable absorbent pad or liner. Those of skill in the art will recognize that many other embodiments and combinations are possible.

### Representative Materials to Further Promote Discretion by Camouflaging, Masking, or Minimizing Sonic and/or Olfactory Signals

In addition to visual queues that may cause others to detect the presence of a disposable absorbent article, sonic or olfactory queues may also signal to others that a user is employing a disposable absorbent article. Accordingly, to further promote discretion, a disposable absorbent article of the present invention may also employ materials that effect noises typical of the noises produced by the second article of manufacture with which the disposable absorbent article will be used. Or a disposable absorbent article of the present invention may employ materials that minimize or reduce noise effected by the disposable absorbent article, or of a component thereof. Typically the second article of manufacture with which the disposable absorbent article is used will include a woven, cloth-like material. Accordingly, soft, cloth-like nonwoven materials may be selected for the liquid-permeable layer.

Polymeric films are often used for the liquid-impermeable layer, however, and such films may produce excessive noise during use (e.g., “rattling” or “rustling” sounds). As noted in U.S. Patent Publication No. 2005/0112338 A1 to Michael Faulks, et al., entitled “Reduced-Noise Composite Materials and Disposable Personal Care Devices Employing Same,” which is hereby incorporated by reference in its entirety in a manner consistent herewith, novel noise-reduction composite materials of the type disclosed therein may be used, for example, as the liquid-impermeable layer. So, for example, elastomeric materials such as polyisoprene, polybutadiene, polyisobutylene, polyurethanes, silicone rubbers, atactic polypropylene, synthetic block co-polymers such as styrene-butadience-styrene (SBS), styrene-isoprene-styrene (SIS), and styrenethylene-butylene-styrene (SIBS) rubbers, and the like may be coated or applied to liquid-impermeable films as described in the referenced publication. The selected noise-reducing coating material may be applied using slot coat, swirl spray, meltblown spray, or other methods of applying coatings well known in the art. Additional details for preparing such noise-reducing composites, which, as noted above, may be used in certain embodiments of the present invention to further promote discretionary use of a disposable absorbent article, are disclosed in the referenced U.S. patent publication.

Other versions of the present invention may employ one or more odor-control materials to mask, minimize, or camouflage the smell of urine and/or other bodily fluids. Such materials include, for example, talc, zeolites, or activated carbon particles (or in fiber form) or silica, opacifiers, graphite, graphite nanoparticles, carbon nanotubes, silica nanoparticles, colloidal metals such as silver or gold, sodium bicarbonate, aluminas, titanias, sodium carbonates, sodium phosphates, zinc and copper sulfates, other chemicals known to control odors, and mixtures thereof. The amount of odor sorbent will vary depending on the effectiveness of the sorbent chosen but should generally be in the range of about 2 to about 80 weight percent, desirably between about 5 and 75 weight percent and more desirably between about 10 and 30 weight percent. A particularly suggested odor sorbent includes, but is not limited to, activated carbon particles. Odor control materials are typically employed in the absorbent core, but may be disposed on or in the liquid-permeable layer, the liquid-impermeable layer, or other components of the disposable absorbent article.

In some versions of the invention, both odor-control materials and noise-reducing materials are used in making the disposable absorbent article comprising a graphic adapted to facilitate discretionary use of said article.

### Representative Articles of Manufacture with which a Disposable Absorbent Article Having a Graphic Disposed Thereon Adapted to Promote Discreet Use May be Used

A disposable absorbent article having a graphic disposed thereon may be adapted to promote discreet use in conjunction with a number of different articles of manufacture that will be proximate or adjacent to said disposable absorbent article during use of said disposable absorbent article. For example, a disposable, absorbent pad or liner comprising a liquid-impermeable layer, an absorbent core, and a liquid-permeable layer may be used adjacent to the seat of a chair, a bed mattress or sheets or covers over the mattress, inside a sleeping bag, as a floor covering, etc. When placed, the liquid-impermeable layer of the disposable, absorbent pad or liner will face the chair, mattress, sleeping bag, or other such article. The liquid-permeable layer will face, and generally will directly contact (or be capable of contacting), the user of the disposable absorbent pad or liner. The graphic that is adapted to facilitate the disposable absorbent article to blend with, be camouflaged by, be the same or similar as, be coordinated with, or have a common theme with, a second graphic disposed on a second article of manufacture will generally be printed on the liquid-permeable layer (or at least be visible when this layer is facing the viewer; i.e., other translucent or transparent layers may be over the layer bearing the graphic, with the graphic still being visible). Alternatively, the graphic may be disposed on the disposable absorbent article by combining dyed fiber, whether the fiber is all of one color, or of different color; i.e., the fiber making up, for example, a nonwoven, liquid-permeable layer may be dyed or colored so that it matches the color of a second article of manufacture with which the disposable absorbent article will be used. Any method may be used to dispose a graphic on the disposable absorbent article so that it matches, blends with, is the same or similar as, is camouflaged by, is coordinated with, or has a common theme with a second graphic on the second article of manufacture with which the disposable absorbent article will be used.

### Representative Media and Methods by which Consumers, Users, and/or Others May be Made Aware of Disposable Absorbent Articles Having a Graphic Disposed Thereon Adapted to Promote Discreet Use

A manufacturing company or retailer may use a number of different media and ways to communicate to consumers that a disposable absorbent article having a graphic disposed thereon is adapted to promote discreet use of said article.

The manufacturer or distributor of a disposable absorbent article of the present invention may fashion messages, statements, or copy to be transmitted to a purchaser, consumer, or user of said disposable absorbent article. Such messages, statements, or copy may be fashioned to help facilitate or establish an association in the mind of a user of the article between a disposable absorbent article of the present invention, or use thereof, and one or more mental states, psychological states, or states of well being. The communication, statements, or copy may include various alphanumeric strings, including, for example: “confidence,” “confident,” “discretion,” “security,” “secure,” “discreet,” “dignity,” “clean,” “fresh,” “health,” “hygiene,” “well,” “well being,” “scent,” “convenient,” or “disposable,” derivatives or combinations thereof, or other such words or statements. In one embodiment, the communication, statements, or copy associate a disposable absorbent article of the present invention and discreet use. In another embodiment, the communication, statements, or copy associate a disposable absorbent article of the present invention preserving dignity. In another embodiment, the communication, statements, or copy associate a disposable absorbent article of the present invention and a registered or common-law trademark, name, brand name, and/or logo of the seller, manufacturer, and/or distributor of the disposable absorbent article. In another embodiment, the communication, statements, or copy associate a disposable absorbent article of the present invention and a registered or common-law trademark, name, brand name, and/or logo of the seller, manufacturer, and/or distributor of a second article of manufacturer that will be proximate or adjacent to the disposable absorbent article during use of the disposable absorbent article (e.g., a disposable absorbent pad or liner, having a first graphic disposed thereon, to be used in conjunction with a sleeping bag—i.e., a second article of manufacture—with the sleeping bag having a second graphic disposed on the interior of the sleeping bag).

Messages, copy, statements, and/or alphanumeric strings like those referred to above may be used either alone, adjacent to, or in combination with, other alphanumeric strings. The communication, statements, message, or copy could take the form of (i.e., be embodied in a tangible medium such as) a newspaper advertisement, a television advertisement, a radio or other audio advertisement, items mailed directly to addressees, items emailed to addresses, Internet Web pages or other such postings, free standing inserts, coupons, various promotions (e.g., trade promotions), co-promotions with other companies, copy and the like, boxes and packages containing the product (in this case an appliance of the present invention), and other such forms of disseminating information to consumers or potential consumers. Other exemplary versions of such communications, statements, messages, and/or copy may be found in, for example, U.S. Pat. Nos. 6,612,846 and 6,896,521, both entitled “Method for Displaying Toilet Training Materials and Display Kiosk Using Same”; co-pending U.S. application Ser. No. 10/831,476, entitled “Method of Enunciating a Pre-Recorded Message Related to Toilet Training in Response to a Contact”; co-pending U.S. application Ser. No. 10/956,763, entitled “Method of Manufacturing and Method of Marketing Gender-Specific Absorbent Articles Having Liquid-Handling Properties Tailored to Each Gender”; each of which is incorporated by reference in their entirety in a manner consistent herewith.

It should be noted that when associating statements, copy, messages, or other communications with a package (e.g., by printing text, images, symbols, graphics, color(s), or the like on the package; or by placing printed instructions in the package; or by associating or attaching such instructions, a coupon, or other materials to the package; or the like) containing one or more disposable absorbent articles of the present invention, the materials of construction of said package may be selected to reduce, impede, or eliminate the passage of water or water vapor through at least a portion of the package. Furthermore, the materials of construction of said package may be selected to minimize or impede the passage of light through said package, including minimizing or impeding the passage of electromagnetic waves of a selected wavelength or wavelengths.

For purposes of this application, “packages,” “containers,” “envelopes,” “bags,” “packets,” and the like are interchangeable in the sense that they refer to any material adapted to enclose and hold either individual disposable absorbent articles (as in, for example, an individual packet containing a single disposable absorbent article), or a plurality of disposable absorbent articles (as in a flexible bag made of film or plastic container containing a plurality of disposable absorbent articles, whether or not each of the individual disposable absorbent articles are enclosed and held in a separate material—such as individual packets).

In some embodiments of the present invention, a package will contain not only one or more disposable absorbent articles of the present invention, but other articles of manufacture. In one embodiment, a disposable absorbent pad or liner of the present invention is sold, transferred, distributed, or marketed with a sleeping bag. It should be noted that such combinations may be marketed and packaged as described in the preceding paragraphs.

Reference now will be made to various embodiments of the invention, examples of which are set forth below. Each example is provided by way of explanation of the invention, not as a limitation of the invention. In fact, it will be apparent to those skilled in the art that various modifications and variations can be made of this invention without departing from the scope or spirit of the invention.

### EXAMPLES

### Prophetic Example and Representative Embodiment 1

### Disposable Absorbent Liner for Sleeping Bag

A Coleman®-brand sleeping bag is obtained. A spectrophotometer, calorimeter, image analysis, and/or other optical device is used to obtain quantitative colorimetric measurements of the interior of the sleeping bag (i.e., the color of the interior is ascertained in this or some other manner, e.g., seeking to match, as closely as possible, the color and/or pattern of the interior of the sleeping bag with standard color tiles or swatches). Conventional techniques are then used to determine dyes, pigments, inks, and/or other additives that may be added to or associated with—e.g., via printing—a liquid-permeable layer and/or materials from which the layer is constructed so that the layer has a color identical or similar to the color of the interior of the sleeping bag (i.e., so that a viewer finds it more difficult to determine that an absorbent liner or pad is present in the sleeping bag, compared to, for example, a disposable absorbent liner or pad that is white if used with a sleeping bag having a colored interior).

A pad comprising a liquid-permeable layer having a color disposed thereon similar or identical to the color and/or pattern of the interior of a Coleman®-brand sleeping bag is then made. The liquid-permeable layer having this color is attached to a liquid-impermeable layer using ultrasonic bonding equipment, with an absorbent core comprising a cellulosic fluff pulp disposed between the layers prior or concurrent to the layers being joined. The dimensions of the disposable absorbent article are selected so that it can be inserted into the sleeping bag.

A pad made as described in the preceding paragraph is sold under a trademark, trade name, and/or logo of the pad manufacturer in a package. A statement disposed on the package associates use of the disposable absorbent article with discretion, security, dignity, confidence, and/or some other like descriptor.

### Prophetic Example and Representative Embodiment 2

### Disposable Absorbent Liner for Sleeping Bag

A manufacturer of a disposable absorbent liner or pad communicates and/or collaborates with a manufacturer of a sleeping bag to ascertain the precise chemistry and pattern of any graphic disposed on the interior of the sleeping bag. The manufacturer of the disposable absorbent liner then makes a disposable absorbent product, such as a disposable absorbent pad or liner, having a graphic disposed thereon matching that of the graphic disposed on the interior of the sleeping bag. The manufacturer of the disposable absorbent liner or pad then markets and sells the disposable absorbent line or pad having the matching graphic to customers under its own logos, brands, trade names, and/or trademarks. Alternatively, the manufacturer of the disposable absorbent liner or pad sells said liner or pad to the manufacturer of the sleeping bag, with the manufacturer of the sleeping bag then selling the line or pad to its customers under its logos, brands, trade names, and/or trademarks.

### Prophetic Example and Representative Embodiment 3

### Kits Comprising a Disposable Absorbent Article Disposed Thereon and a Second Article of Manufacture Having a Second Graphic Disposed Thereon, Wherein the First Graphic Matches, Blends With, is Camouflaged by, is the Same or Similar as, or has a Common Theme with the Second Graphic

A disposable absorbent pad or liner as described in either Example 1 or 2 above is sold together with a sleeping bag. As discussed above, the graphic disposed on the absorbent pad or liner is the same or similar to the graphic disposed on the interior of the sleeping bag. This kit, collection, or bundle is sold under the trade name, trademark, brand, and/or logo of: the manufacturer of the pad or liner; the manufacturer of the sleeping bag; the retailer of the kit, collection, or bundle; or some combination thereof.

### Prophetic Example and Representative Embodiment 4

### Product Line Comprising a Plurality of SKUs or Other Designators, with Each SKU or Other Designator Corresponding to a Disposable Absorbent Pad or Liner Having a Different Graphic Disposed Thereon

A manufacturer of disposable absorbent products, using approaches such as those described in Examples 1 and 2 above and elsewhere in this application, develops and markets a product line comprising a plurality of SKUs or other designators. Each SKU or designator corresponds to a disposable absorbent pad or liner with a different graphic. A consumer is able to select that SKU or designator corresponding to a graphic that will match, or be the same or similar as, a second graphic disposed on the interior of a sleeping bag with which the consumer or other user will use the selected disposable absorbent liner or pad.

### Prophetic Example and Representative Embodiment 5

### Packages for a Product Line Comprising a Plurality of SKUs or Other Designators, with Each SKU or Other Designator Corresponding to a Disposable Absorbent Pad or Liner Having a Different Graphic Disposed Thereon

Each SKU or designator corresponds to a package comprising a statement disposed in or on said package. The statement is adapted to communicate to a consumer or user that the consumer or user may select that disposable absorbent pad or liner having a graphic that is the same or similar to a second graphic disposed on a second article of manufacture with which the consumer or user will use the disposable absorbent pad or liner.

The above examples are representative. While a number of the prophetic examples identified the disposable absorbent article as a pad or liner, and the second article of manufacturer as a sleeping bag, other disposable absorbent articles and second articles of manufacturer may be used.

We claim:

1. A line of disposable absorbent products adapted to promote discreet use, the product line comprising:
a plurality of SKUs, wherein each SKU corresponds to a disposable absorbent product having a graphic disposed thereon, and wherein each SKU corresponds to a different graphic; and
a statement embodied in a tangible medium proximate to one or more disposable absorbent products in the product line, wherein the statement associates use of the disposable absorbent product with one or more of “confidence,” “confident,” “discretion,” “security,” “secure,” “discreet,” “dignity,” “clean,” “fresh,” “health,” “hygiene,” “well,” “well being,” “scent,” “convenient,” or “disposable.”

2. The line of claim 1 wherein the disposable absorbent product is a disposable absorbent pad adapted to be used with furniture, a bed, a sleeping bag, or a floor.

3. A line of disposable absorbent products adapted to promote discreet use, the product line comprising:
a plurality of SKUs, wherein each SKU corresponds to a disposable absorbent product having a graphic disposed thereon, and wherein each SKU corresponds to a different graphic;
a plurality of packages, wherein each package contains a selected number of disposable absorbent products corresponding to a given SKU within the product line; and
a statement disposed on, in, or proximate to the package, wherein the statement instructs a potential purchaser of the package to select a disposable absorbent product having a graphic disposed thereon that is the same or similar to a second graphic disposed on a second article of manufacture that will be proximate to the disposable absorbent product during use of said disposable absorbent product.

4. The product line of claim 3 wherein the disposable absorbent products are disposable absorbent pads or disposable absorbent liners for sleeping bags.

5. The product line of claim 3 wherein the disposable absorbent products are disposable absorbent pads for beds.

6. The product line of claim 3 wherein the disposable absorbent products are disposable absorbent pads for furniture.

7. The product line of claim 3 wherein the disposable absorbent products are disposable absorbent floor coverings.

8. A kit for promoting discreet use of a disposable absorbent article, the kit comprising:
a disposable absorbent article having a first graphic disposed on said article; and
a second article of manufacture having a second graphic disposed on said second article of manufacture, wherein the first graphic and the second graphic are the same or similar.

9. The kit of claim 8 wherein the disposable absorbent article is a disposable absorbent undergarment and the second article of manufacture is a sleeping bag liner.

10. The kit of claim 8 wherein the disposable absorbent article is a disposable absorbent pad and the second article of manufacture is an undergarment.

11. The kit of claim 8 wherein the disposable absorbent article is a disposable absorbent pad and the second article of manufacture is a sleeping bag.

12. The kit of claim 8 wherein the disposable absorbent article is a disposable absorbent pad and the second article of manufacture is a bed sheet.

13. The kit of claim 8 further comprising a package containing the disposable absorbent article and the second article of manufacture, wherein a statement disposed on, in, or proximate to said package associates use of the kit with discretionary use of said disposable absorbent article due in part to the first graphic and second graphic being the same or similar.

14. A package comprising:
a disposable absorbent article comprising:
a component comprising a first graphic disposed thereon that is adapted to be the same or similar to a second graphic on a second article of manufacture that will be proximate to said disposable absorbent article during use of said disposable absorbent article; and
a container containing said disposable absorbent article, wherein a statement disposed in, on, or proximate to said container associates use of the disposable absorbent article with discretionary use of said disposable absorbent article due in part to the first graphic and second graphic being the same or similar.

15. The package of claim 14 wherein the component is a liquid-permeable layer.

16. The package of claim 14 wherein the component is a liquid-impermeable layer.

17. The package of claim 14 wherein the disposable absorbent article further comprises a liquid-impermeable layer comprising a noise-control material.

18. The package of claim 14 or 17 wherein the disposable absorbent article further comprises an absorbent core comprising an odor-control agent.

19. The package of claim 14 wherein the container includes the name and/or logo of the manufacturer of the disposable absorbent article.

20. The package of claim 14 wherein the container includes the name and/or logo of the seller of the disposable absorbent article.

21. The package of claim 14 wherein the container includes the name and/or logo of the manufacturer of the second article of manufacture.

22. A component of a disposable absorbent article, wherein the component includes a first graphic disposed thereon adapted to be the same or similar to a second graphic on a second article of manufacture that will be proximate to said component of a disposable absorbent article during use of said disposable absorbent article, and wherein a message embodied in a tangible medium associates use of the disposable absorbent article employing said component with discretionary use of said disposable absorbent article due in part to the first graphic and second graphic being the same or similar.

23. A package comprising:
a disposable absorbent article comprising:
a liquid-permeable layer having a first graphic disposed thereon adapted to be the same or similar to a second graphic on a second article of manufacture that will be proximate to said disposable absorbent article during use of said disposable absorbent article;
a liquid-impermeable layer comprising a noise-control material, wherein at least a portion of the liquid-impermeable layer is attached to said liquid-permeable layer; and
an absorbent core comprising an odor-control agent, wherein the absorbent core is positioned between the liquid-permeable layer and the liquid-impermeable layer; and
a container containing said disposable absorbent article, wherein a statement disposed in, on, or proximate to said container associates use of the disposable absorbent article with discretionary use of said disposable absorbent article due in part to the first graphic and second graphic being the same or similar.
